(19) United States
(12) Patent Application Publication
(10) Pub. No.: US 2026/0306755 A1
(43) Pub. Date: Oct. 8, 2026

(54) SYSTEM, METHOD, AND APPARATUS FOR MITIGATING THE RISE OF INFUSION-INDUCED HYPOTHERMIA

(71) Applicant: TIER ONE SYSTEMS LLC, Lafayette, CA (US)

(72) Inventor: Marc Lurie, Lafayette, CA (US)

(21) Appl. No.: 19/702,422

(22) Filed: Jun. 9, 2026

Related U.S. Application Data
(63) Continuation of application No. 19/429,792, filed on Dec. 22, 2025.
(60) Provisional application No. 63/737,319, filed on Dec. 20, 2024.
(60) Provisional application No. 63/751,071, filed on Jan. 29, 2025.
(60) Provisional application No. 63/758,126, filed on Feb. 13, 2025.
(60) Provisional application No. 63/766,066, filed on Mar. 3, 2025.

(51) Int. Cl. A61M 5/44

(52) U.S. Cl. CPC A61M 5/445

(57) ABSTRACT

Devices, systems, and methods for mitigating the risk of infusion-induced hypothermia are disclosed. In an example, a warming pouch includes a sleeve to hold a container of fluid for infusion into a patient. The warming pouch comprises a warming pocket housing a reactant. An activation solution is housed in a separate activation pocket, which is fluidically coupled to the warming pocket. A frangible seal may be positioned between the warming pocket and the activation pocket. Breakage of the frangible seal allows for intermixing of the reactant and the activation solution, thereby starting the exothermic reaction. The warming pouch is configured to surround the container of fluid throughout the exothermic reaction, evenly distributing heat throughout the fluid prior to and during infusion of the fluid to the patient.

### PRIORITY CLAIM

The present application is a continuation application of U.S. patent application Ser. No. 19/429,792, filed Dec. 22, 2025, which claims priority to and the benefit of the following provisional patent applications: U.S. Provisional Patent Application Ser. No. 63/766,066, filed on Mar. 3, 2025, U.S. Provisional Patent Application Ser. No. 63/758,126, filed on Feb. 13, 2025, U.S. Provisional Patent Application Ser. No. 63/751,071, filed on Jan. 29, 2025, and U.S. Provisional Patent Application Ser. No. 63/737,319, filed on Dec. 20, 2024, the entirety of which are incorporated herein by reference.

### BACKGROUND

In the immediate aftermath of a severe traumatic injury resulting in blood loss, such as in a battlefield, a patient may enter hypovolemic shock. During this period, the patient is at high risk for hypothermia, acidosis, and coagulopathy—known as the “Lethal Triad.” These three interrelated conditions form a compounding positive feedback loop that can result in organ damage or death without direct and immediate medical intervention.

A recommended pre-hospitalization procedure in this situation is to administer one or more units of whole blood. However, blood products are stored at approximately 2 to 6 °C, while the temperature of the human body is approximately 37 °C. As a result of this temperature difference, although it mitigates the risk of death from coagulopathy and acidosis, the very act of infusing blood into a patient increases the risk of death from the third leg of the lethal triangle—hypothermia.

In attempts to address this need in the context of point-of-injury care, manufacturers have released a variety of battery-operated inline warming devices. Described broadly, battery-operated inline warming devices operate by applying an electrical current to a metallic warming surface associated with an intravenous administrative set. Blood or other fluid is warmed by contact with the warming surface as it flows through the administrative set. Such devices are notorious among medics and other healthcare professionals for poor warming output, a problem that becomes increasingly acute at higher flow rates. Other drawbacks include biocompatibility risks due to blood contact and complexity of operation.

Additional drawbacks apply in a military context. Battery-operated inline warmers are extremely vulnerable to environmental conditions. In warm climates, such as deserts, known inline warmers shutdown to prevent thermal runaway. Conversely, in cold climates, even those above freezing, known inline warmers deplete their batteries quickly and the length of the runtime collapses due to the effects of cold temperatures on battery chemistry. As a result, the U.S. military currently has no known all-weather blood and fluid warming capability.

In addition to environmental susceptibility, battery-operated inline warmers are bulky and heavy, typically adding 1 to 2 kilograms of weight to a medic bag. This is a relatively enormous amount of mass as medics typically count loadouts to the gram. Besides increasing the load and physical toll on the medic over a multi-day mission, this extra bulk and weight also means that other life-saving supplies are potentially sacrificed to create room for battery-operated inline warmers, including their consumables and spare batteries.

Moreover, due to the nature of war, often multiple victims need blood simultaneously. However, due to their cost, bulk, and weight, only a single battery-operated inline warmer is carried by a medic in the battlefield. This means that blood is limited to being warmed sequentially, one unit and one patient at a time, instead of concurrently across multiple units and multiple patients. And even this overstates the case as many battery-operated warmers cannot warm two full units of blood outside of idealized laboratory conditions.

The proprietary batteries incorporated into these systems also require recharging, which in turn requires access to a stable continuous power supply, a dependency that does not exist in the battlefield. As such, these systems may be useful to warm or partially warm one or two units of blood in the field, and thereafter constitute a non-performing medical asset for the medic to carry throughout the mission.

From the perspective of supply-chain safety and reliability, although technically manufactured in the United States (or its allies), most and perhaps all battery-operated warming devices source components from potential adversaries, which puts the supply chain at risk during conflict or impending conflict.

Finally, regardless of their origin, the electronics underlying these warmers remain fragile and susceptible to breakage in the field. The electronics also typically fail EMI/EMC testing conducted to determine whether electromagnetic emissions produced by these warming devices will affect aircraft navigation and communication equipment or, conversely, be susceptible to the fields generated by the aircraft equipment. These same emissions provide a potential means for an adversary to detect usage and location in a battlefield.

Battery-operated inline warmers are, in the end, complex computer-controlled systems that attempt to solve a problem that is better and more reliably addressed through a chemically-powered solution that warms an entire unit of fluid in a single operation. An all-weather, ultralight, chemically-powered fluid warmer is accordingly needed.

### SUMMARY

The present disclosure provides a new and innovative apparatus/device, system, and method to warm whole blood, blood components, crystalloids, colloids, and other fluids for mitigating the risk of infusion-induced hypothermia. As disclosed herein, a warming pouch apparatus/device is configured to contain a reactant. An activation solution is introduced to the reactant, generating an exothermic reaction for a desired time. Heat from the exothermic reaction is used to bring a container of fluid to a target temperature range. Steam and gaseous byproducts generated during the exothermic reaction vent from the warming pouch to an exterior atmosphere, thereby preventing excess pressure from building within the warming pouch.

Example systems, methods, and devices/apparatuses are disclosed herein to mitigate the risk of infusion-induced hypothermia. The example systems, methods, and devices/apparatuses are configured to produce an exothermic reaction to warm a container of fluid. Some fluids, such as blood, must be refrigerated in storage to avoid negative effects such as spoilage. Other fluids, such as saline, are stored at ambient temperatures typically warmer than refrigerated fluids yet still below human (or other animal) body temperature. However, inserting any cold fluid into the human body (or a body of any animal) may introduce the risk of hypothermia. Therefore, a container of fluid may need to be heated prior to insertion of the fluid into a human body. After identifying the container of fluid needing its temperature raised, the disclosed systems, methods, and devices/apparatuses are configured to produce an exothermic reaction in a warming pouch. The process is instigated by an operator. As disclosed herein, the exothermic reaction is caused by introducing an activation solution to a reactant housed in a warming pouch. Intermixing the activation solution and the reactant causes an exothermic reaction to begin, producing heat and gaseous byproducts. The gaseous byproducts escape to an exterior atmosphere through one or more vents defined in the disclosed warming pouch. The container of fluid is positioned within or otherwise against at least one side of the warming pouch to allow heat to transfer from the exothermic reaction within the warming pouch to the fluid within the container.

In some embodiments, the activation solution is stored separated from the warming pouch. Storing the activation solution separately from the warming pouch reduces the potential risk of accidental activation of the exothermic reaction prior to the point of anticipated use. In an embodiment, the disclosed warming pouch is double-sided. The sides of the warming pouch are bonded together at or near the edges to form a body comprising a sleeve with a cavity sized to house a container of fluid, an open end to receive a container of fluid, and a second opening running between the body and the cavity to allow for access to the container of fluid. In such embodiment, each side includes an interior flexible wall and an exterior flexible wall bonded at or near the edges. The interior flexible walls are configured to define the cavity and abut a container of fluid. Each bonded set of interior and exterior walls (for each side) are configured to contain or otherwise enclose a warming pocket containing a reactant. A vent is provided at or near a top of each side to enable steam and/or other gaseous byproducts generated from the exothermic reaction to flow to an exterior atmosphere.

In such embodiment, an operator initiates the exothermic reaction by introducing an activation solution to the reactant, which initiates the exothermic reaction. In an example, the operator introduces the activation solution through a vent, such as by injection. The exothermic reaction produces heat as the reaction occurs, which in turn warms a container of fluid placed in the cavity of the warming pouch. In this embodiment, the exothermic reaction produces gaseous byproducts. Pressure from these gaseous byproducts communicate to the exterior atmosphere though the one or more vents.

In another embodiment, the present disclosure includes a warming pouch that includes both the activation solution and the reactant. In such embodiments, the two sides of the warming pouch are bonded together at or near the edges to form a body, comprising a sleeve with a cavity sized to house a container of fluid, an open end to receive a container of fluid, and a second opening running between the body and the cavity to allow for access to the container of fluid. In such an embodiment, each side includes an interior flexible wall and an exterior flexible wall bonded at or near the edges. The interior flexible walls are configured to define the cavity and abut a container of fluid placed therein. Each bonded set of interior and exterior walls are configured to contain a stacked set of pockets: a lower warming pocket containing reactant and an upper activation pocket containing an activation solution. The warming pocket and the activation pocket are separated by a first frangible seal. Above the activation pocket is a vent to the exterior atmosphere. The activation pocket and the vent are separated by a second frangible seal.

In this embodiment, to begin the exothermic reaction an operator may fold the top of each side of the warming pouch downwards and squeeze in order to apply pressure to the activation pocket. The accumulation of pressure on the activation pocket eventually causes the first frangible seal to break, thereby enabling the activation solution to flow downward and mix with the reactant to initiate an exothermic reaction. The exothermic reaction produces heat as the reaction occurs, which in turn warms a container of fluid placed in the cavity of the warming pouch. Even after bulk of the exothermic reaction completes, the container of fluid may continue to receive heat from residual energy introduced into the container from the warming pouch as residual reactant mixes with the activation solution. In this embodiment, the exothermic reaction produces gaseous byproducts. The pressure generated from the accumulation of the gaseous byproducts increases over time eventually causing the second frangible seal to breach between the activation pocket and the one or more vents, thereby enabling the gaseous byproducts to escape into the exterior atmosphere.

In some embodiments useful for when less warmth is desired, such as warming saline from room temperature to a body temperature for example, only one side of the warming pouch is configured to provide warmth. In these embodiments, the warming pouch is configured to contain a warming pocket on only one side. In such embodiments, the opposite side of the warming pouch may be a single flexible wall configured to form one half of the cavity and abut a container of fluid, instead of a flexible interior wall and flexible exterior wall.

To increase the safety of processing the reactant, in some embodiments the reactant may be housed in a warming pad comprised of a plurality of sheets of gas and water permeable material bonded to each other to form a chamber. The chamber houses the reactant. The warming pad is inserted and sealed in the warming pouch at the time of manufacture. To simplify the loading of the warming pad of the device, the warming pad is inserted into an unbonded section of the side of the warming pocket during manufacture, which such unbonded section then sealed and bonded. In another embodiment without an activation pocket, the warming pad is inserted down through an unbonded section at the top of interior wall and exterior wall. In some instances, a frangible seal may be created above the warming pad.

In light of the disclosure herein and without limiting the disclosure in any way, in a first aspect of the present disclosure, which may be combined with any other aspect listed herein, a fluid warming pouch includes a body having a plurality of sides, each side comprising an interior flexible wall and an exterior flexible wall. The interior flexible wall is configured to abut a container of fluid. The interior flexible wall and the exterior flexible wall are bonded to each other. Each side includes a warming pocket and a vent to an exterior atmosphere. The warming pocket houses or otherwise encloses a reactant configured to react with an activation solution introduced from an external source. A vent is fluidically coupled to the warming pocket.

In a second aspect of the present disclosure, which may be combined with any other aspect listed herein, the warming pocket is the first warming pocket of a plurality of warming pockets housing the reactant.

In a third aspect of the present disclosure, which may be combined with any other aspect listed herein, the warming pocket is depressurized.

In a fourth aspect of the present disclosure, which may be combined with any other aspect listed herein, the warming pocket contains a warming pad. The warming pad includes a plurality of sheets of gas and water permeable material bonded to each other forming a chamber that contains or encloses the reactant.

In a fifth aspect of the present disclosure, which may be combined with any other aspect listed herein, the chamber included in the warming pad is a first chamber in a plurality of chambers that each contain the reactant.

In a sixth aspect of the present disclosure, which may be combined with any other aspect listed herein, the vent is the first vent of a plurality of vents.

In an seventh aspect of the present disclosure, which may be combined with any other aspect listed herein, the vent is defined by an aperture in the interior flexible wall, the exterior flexible wall, or any combination thereof.

In an eighth aspect of the present disclosure, which may be combined with any other aspect listed herein, the vent is positioned between the interior flexible wall and the exterior flexible wall. The vent is defined by an aperture created by an unbonded portion of the interior flexible wall and an unbonded portion of the exterior flexible wall. The vent is located at a top of the fluid warming pouch when the fluid warming pouch is positioned for use.

In a ninth aspect of the present disclosure, which may be combined with any other aspect listed herein, the warming pouch includes a tab affixed to the body. The tab defines an aperture from which the fluid warming pouch can be hung.

In a tenth aspect of the present disclosure, which may be combined with any other aspect listed herein, the tab is the first tab of a plurality of tabs.

In an eleventh aspect of the present disclosure, which may be combined with any other aspect listed herein, the body is in a shape of a sleeve defining an open end to receive the container of fluid. The sleeve defines a cavity sized to house the container of fluid.

In a twelfth aspect of the present disclosure, which may be combined with any other aspect listed herein, the body further defines a second opening to allow a connector or a port of the container of fluid to pass through.

In a thirtieth aspect of the present disclosure, which may be combined with any other aspect listed herein, the warming pocket is configured to receive the activation solution from an external source via injection.

In a fourteenth aspect of the present disclosure, which may be combined with any other aspect listed herein, the activation solution is chosen from the group of water, isotonic saline, hypertonic saline, or a combination thereof.

In a fifteenth aspect of the present disclosure, which may be combined with any other aspect listed herein, the activation solution contains an antifreeze additive.

In a sixteenth aspect of the present disclosure, which may be combined with any other aspect listed herein, the antifreeze additive is calcium chloride (CaCL₂).

In a seventeenth aspect of the present disclosure, which may be combined with any other aspect listed herein, the activation solution is provided as a 28% calcium chloride solution.

In an eighteenth aspect of the present disclosure, which may be combined with any other aspect listed herein, the reactant is chosen from the group of a mixture of Magnesium and Iron (Mg—Fe), a mixture of aluminum chloride and calcium oxide (AlCl₃—CaO), or a mixture of diphosphorus pentoxide and calcium oxide (P₂O₅—CaO).

In a nineteenth aspect of the present disclosure, which may be combined with any other aspect listed herein, the exothermic reaction comprises a warming cycle and a runtime cycle. During the warming cycle, the exothermic reaction causes the fluid within the fluid container to warm and approach a target temperature. During the runtime cycle, the exothermic reaction expends a lower amount of energy to stay within a target range of temperatures for a predetermined period.

In a twentieth aspect of the present disclosure, which may be combined with any other aspect listed herein, the minimum target temperature for the fluid within the fluid container is approximately ten degrees Celsius. The target range is between approximately ten and forty-six degrees Celsius.

In a twenty-first aspect of the present disclosure, which may be combined with any other aspect listed herein, the warming cycle is within a period of three minutes. The runtime cycle is a minimum of ten minutes.

In a twenty-second aspect of the present disclosure, which may be combined with any other aspect listed herein, the fluid to be warmed is whole blood, one or more blood products, a crystalloid, a colloid, or water.

In a twenty-third aspect of the present disclosure, which may be combined with any other aspect listed herein, the reactant is a powder comprising a plurality of particulate sizes.

In a twenty-fourth aspect of the present disclosure, which may be combined with any other aspect listed herein, the body is composed of plastic, foil, plasticized foil, laminated foil or any combination thereof.

In a twenty-fifth aspect of the present disclosure, which may be combined with any other aspect listed herein, the interior flexible wall is composed of a material with a first thermal conductivity. The exterior flexible wall is composed of a material with a second thermal conductivity. The first thermal conductivity is greater than the second thermal conductivity.

In a twenty-sixth aspect of the present disclosure, which may be combined with any other aspect listed herein, the body includes one side having the interior and exterior flexible walls and an opposing side with a single wall.

In a twenty-seventh aspect of the present disclosure, which may be combined with any other aspect listed herein, a fluid warming pouch includes a body having a plurality of sides, each side comprising an interior flexible wall and an exterior flexible wall. The interior flexible wall is configured to abut a container of fluid. The interior flexible wall and the exterior flexible wall are bonded to each other. Each side includes a warming pocket, an activation pocket, a first frangible seal, a second frangible seal, and a vent to the exterior atmosphere. The warming pocket houses a reactant. The activation pocket houses an activation solution. The activation pocket is fluidically coupled to the warming pocket. The first frangible seal is positioned between the warming pocket and the activation pocket. Breakage of the first frangible seal creates a channel between the warming pocket and the activation pocket allowing for intermixing of the reactant and the activation solution to generate heat via an exothermic reaction. The vent is fluidically coupled to at least one of the warming pocket, the activation pocket, or any combination thereof. The second frangible seal is positioned between the vent and at least one of the warming pocket, the activation pocket, or any combination thereof. Breakage of the second frangible seal creates a channel between at least one of the warming pocket, the activation pocket, or any combination thereof and the exterior atmosphere allowing for gaseous byproducts of the exothermic reaction to escape to the exterior atmosphere. In a twenty-eighth aspect of the present disclosure, which may be combined with any other aspect listed herein, the first frangible seal is configured to break within a first range of pressures.

In a twenty-ninth aspect of the present disclosure, which may be combined with any other aspect listed herein, the second frangible seal is configured to break within a second range of pressures, wherein the second range of pressures is lower than the first range of pressures.

In a thirtieth aspect of the present disclosure, which may be combined with any other aspect listed herein, the activation pocket is the first activation pocket in a plurality of activation pockets housing the activation solution.

In a thirty-first aspect of the present disclosure, which may be combined with any other aspect listed herein, the activation pocket is depressurized.

In light of the present disclosure and the above aspects, it is therefore an advantage of the present disclosure to mitigate the risk of infusion-induced hypothermia.

Additional features and advantages of the disclosed system, methods, and apparatus are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

The inventive subject matter may be understood from reading the following description of non-limiting embodiments, with reference to the attached drawings, wherein below: FIGS. 1A and 1B show a fluid warming pouch, according to an example embodiment of the present disclosure.

FIGS. 2A and 2B show an embodiment of the warming pouch with a seal, according to an example embodiment of the present disclosure.

FIGS. 3A, 3B, and 3C show an example use of the warming pouch, according to an example embodiment of the present disclosure.

FIGS. 4A and 4B show an embodiment of the warming pouch where an activation solution is sealed in an activation pocket at the time of manufacture, according to an example embodiment of the present disclosure.

FIG. 5 shows an embodiment of the warming pouch where vents are removed, according to an example embodiment of the present disclosure.

FIGS. 6A and 6B show an embodiment of a warming pad of the warming pouch, according to an example embodiment of the present disclose.

FIGS. 7A and 7B show different ways of inserting the warming pad of FIGS. 6A and 6B into the warming pouch, according to example embodiments of the present disclosure.

FIG. 8 shows an embodiment where the warming pouch instead includes partitions, according to an example embodiment of the present disclosure.

### DETAILED DESCRIPTION

The disclosed warming pouch is configured to utilize an exothermic chemical reaction to warm a bag of blood, saline, or other fluid in a chemically-powered operation to advance point-of-injury care, such as in a battlefield or other ambulatory environment. While reference is made herein to blood and saline, it should be appreciated that the disclosed warming pouch may be used for any fluid including whole blood, blood products, crystalloids, colloids, total parenteral nutrition solution, dialysis fluid, intravenous medication, water, etc.

In an embodiment, the disclosed warming pouch is a double-sided foldable warming pouch. Opposing sides of the warming pouch are bonded together at or near edges of the at least two sides to form a body. The disclosed body of the warming pouch comprises a sleeve with a cavity sized to house a container of fluid, a first open end to receive the container of fluid, and a second open end located between the body and the cavity to allow for access to the container of fluid. In an example, the container of fluid includes a connector or port that fits through the second open end of the body to allow a fluid tube, such as an intravenous (“IV”) or administrative line, to be connected thereto. As disclosed herein, the first open end is positioned at a top of the body when the warming pouch is positioned for use. Additionally, the second open end is positioned at a bottom of the body when the warming pouch is positioned for use.

In an embodiment, each side of the warming pouch includes an interior flexible wall and an exterior flexible wall bonded at or near the edges. The interior flexible wall is configured to define a portion of the cavity and abut a container of fluid therein. Each bonded set of interior and exterior walls are configured to contain a warming pocket containing reactant.

At or near the top of each side is a vent to an exterior atmosphere. At or near the top is a tab with an aperture to enable the disclosed warming pouch to hang from an IV pole or similar point of attachment.

In an embodiment, in order to promote the rapid and more even distribution of heat and prevent leakage, at least one of the interior flexible wall, the exterior flexible wall, or any combination thereof are comprised of plastic, foil, plasticized foil, laminated foil or any combination thereof.

In an embodiment, the present disclosure is entirely self-contained with a double-sided warming pouch. In this embodiment, the two sides of the warming pouch are bonded together at or near the edges to form a body, comprising a sleeve with a cavity sized to house a container of fluid.

In an embodiment, each side of the body includes an interior flexible wall and an exterior flexible wall. The interior flexible wall is configured to abut a container of fluid. In another embodiment, only one side of the body of the warming pouch comprises an interior flexible wall and an exterior flexible wall and another, opposing side of the body comprises a single flexible wall. In this other embodiment, the single flexible wall and the single interior flexible wall of the warming pouch are configured to define the cavity and abut or otherwise come into contact with a container of fluid.

In an embodiment, the flexible walls of the body comprise plastic or a similar material to prevent leakage of the activation solution and reactant, as discussed herein. In an alternate embodiment, at least one of the interior flexible wall and the exterior flexible wall are composed of plasticized foil or a similar material. The use of foil increases the thermal conductivity of the wall, thereby allowing released energy to be distributed more rapidly and efficiently throughout the disclosed warming pouch. The plastic both protects the foil and prevents leakage of the activation solution and reactant, as discussed herein. In an embodiment, only one of the interior and exterior flexible walls is composed of plasticized foil or similar material and the other wall is composed of plastic or a similar material.

In an embodiment, each side of the warming pouch contains a matched pair of stacked pockets: an upper “activation pocket” containing an activation solution and a lower “warming pocket” containing a reactant. In such an embodiment, each pair of activation and warming pockets is separated by a first frangible seal to prevent communication until ready for use. In an embodiment, to ease manufacturing and promote the rapid communication of the activation solution throughout the reactant, the reactant is a powered mixture contained within a warming pad, which is placed within the activation pocket, as described herein. The warming pad includes a plurality of sheets of gas and water permeable material bonded to each other forming a chamber that contains or encloses the reactant.

In an embodiment, near the top of each warming pouch are a multiplicity of apertures or holes that extend through the exterior wall, the interior wall, or any combination thereof, to vent steam and gas byproducts generated during an exothermic reaction. In an embodiment, there are four evenly spaced rounded rectangular vents with no vent placed directly under one or more plastic tabs with holes described herein. In an alternate embodiment, one or more vents are instead formed from one or more unbounded areas between the interior and exterior flexible walls at the top of the warming pouch (when positioned for use). Below the vents and above the activation pocket is a second frangible seal that prevents leakage of the activation solution into the exterior atmosphere.

To enable its use with the existing tools and procedures that medics are already experienced and comfortable with in the field, the overall geometry of the disclosed warming pouch, when loaded with a container of fluid, may allow it to slide into existing off-the-shelf pressure infusion cuffs. In an embodiment, rounded corners at the bottom of the warming pouch are configured to promote easy loading into mesh and other fabrics utilized in the manufacture of pressure infusion cuffs.

A notched opening at the otherwise bonded bottom edge of the two sides of the warming pouch provides easy access to ports or connectors of a fluid bag. In an example, this notch has a hemispherical shape to promote access to the ports of the fluid bag. Located at the top of the device, at least one plastic tab with an aperture that allows for the warming pouch to be hung during use or transport from, for example, a pole or a carabiner.

To load the warming pouch, an operator slides a fluid bag into an opening between the two sides at the top. A bottom edge of the two sides of the device is at least partially bonded to prevent the fluid bag from sliding all the way through a sleeve of the warming pouch. An unbonded portion of the bottom edge allowed a port or connector of a fluid bag to extend therethrough for external access by an operator for connection to a fluid line. The unbonded portion may be located in a middle of the bottom edge when the warming pouch is positioned for use. Optionally, the operator may then insert the warming pouch, loaded with a fluid bag. into a pressure infusion cuff.

In an embodiment to initiate warming, an operator rolls or folds the top of the warming pouch down approximately 1-3 centimeters past the second frangible seal and squeezes. This squeezing applies pressure to the activation pocket, which contains an activation solution. Because the second frangible seal is nested within rolled or folded material, the applied pressure primary affects the first frangible seal, causing it to breach or otherwise break. The breach of the first frangible seal enables the activation solution to flow downward to mix with the reactant in the warming pocket to initiate warming.

In an embodiment, after mixing the activation solution with the reactant of the warming pocket, the warming pouch enters a period of rapid energy release (e.g., a “warming cycle”) to reach a minimum target temperature. In an embodiment, the configuration of the reactant and the activation solution causes the warming cycle to last five minutes or less to reach a target temperature of approximately 10 °C. In another embodiment, the configuration of the reactant and the activation solution causes the warming cycle to last two to three minutes and reach a minimum target temperature of approximately 20 °C. While faster heating and/or higher minimum target temperatures are technically possible, in the case of whole blood and blood products, achieving such goals increases the risk of causing damage by application of excessive heat to the fluid in contact with the walls of the fluid bag. Conversely, warming cycles longer than two to three minutes create the risk of damage to a patient by delaying treatment. Thus for whole blood and product products, while a wide range of values are technically possible, for battlefield and many other purposes an ideal time range for the warming cycles is between two to three minutes, which has the additional benefit of being the typical amount of time needed by an operator to prepare an administrative set and obtain intravenous access.

During the warming cycle, pressure builds in the now communicating activation and warming pockets. As this pressure builds, an increasing amount of force is applied to the second frangible seal, which is located underneath the vents at the top of the warming pouch. Eventually the increase in pressure causes the second frangible seal to breach or otherwise break and release the generated byproducts (primarily steam and gas) into the atmosphere.

To prevent steam burns, the operator may utilize at least one plastic tab with a hole at the top to suspend the warming pouch from a litter pole, an IV pole, or other apparatus until the warming cycle completes. Alternatively, when the warming pouch is used in conjunction with a pressure infusion cuff, air bladders contained within the cuff provide sufficient insulation to allow the operator to hold and manipulate as a single unit the cuff and the disclosed warming pouch within.

In an embodiment, after the warming cycle completes, the warming pouch transitions from rapid warming to instead expending a lower amount of energy to maintain the temperature of the unit of fluid within a target range (the “runtime cycle”). In some embodiments, the warming pouch may produce negligible amounts of heat during the runtime cycle. In such embodiments, the fluid within the bag may stay within the target range due primarily to residual heat generated during the runtime cycle and communicated from the warming pouch to the fluid bag during the warming cycle. Practitioners skilled in the art realize that different time ranges are possible for the runtime cycle. Unless a present emergency demands faster infusion, a common flow rate for a unit of blood or saline is 50 mL/min, which translates to ten minutes to infuse a full bag of fluid into a patient. As such, practitioners skilled in the art realize that a minimum runtime of ten minutes is required.

In an embodiment, the reactant is a powdered mixture that helps regulate both the warming and runtime cycles. The powdered mixture of the reactant contains particles of a plurality of particulate sizes. In an example, the powdered mixture contains a subset of smaller particulates that react more quickly with the activation solution to generate near-term energy to power the warming cycle. In this same example, the powdered mixture also contains larger particulates that react more slowly with the activation solution, releasing energy over a longer period of time to energize the full runtime cycle. It should be appreciated that the ratio of particulate sizes can be adjusted to control the ratio of time for the warming and runtime cycles.

Accidental activation is a risk to be considered, both to preserve functionality for when needed and to avoid creating unintentional hazards. For example, an operator may land on their assault pack after a parachute drop with items in the assault pack temporarily compressed. To address the risk of accidental activation from compression, in an embodiment the activation and warming pockets are oversized for the contents and depressurized. To further reduce the risk of accidental activation, in an embodiment, the second frangible seal requires less force to breach than the first frangible seal in order to allow the activation solution to communicate into the exterior atmosphere instead of flowing into the warming pocket and activating the warming cycle when accidentally compressed. In an example, the second frangible seal is configured to break with half or three-quarters the force compared to the force needed to break the first frangible seal.

To promote efficiency in patient care, during the warming cycle, an operator prepares the administration set and obtains intravenous access, just as they would with non-warmed fluids. Optimally at the end of the warming cycle, although it may begin earlier at the operator's discretion, the operator begins the flow of blood or other fluids using known steps for an intravenous infusion. Again, just as they normally would, the operator occasionally adjusts pressure on the optional pressure infusion cuff to maintain a needle in a green zone as the fluid bag depletes. The entire process is designed to be simple and easy to follow for an operator, even during moments of acute stress.

The warming process disclosed herein is similar for other non-blood fluids, such as crystalloids, with saline as an example, with one variation: unlike whole blood, which is recommended to be stored at about 5 °C, saline is stored at ambient temperatures. As such, the starting temperature for warming saline and other fluids may often be higher than for cold-stored whole blood (and blood products). Due to the properties of thermodynamics, the rate of temperature increase tends to be lower for a fluid with a higher initial temperature than for a fluid with a lower initial temperature and, up to a limit, the average temperature for both fluids tends to converge during the warming operation. For example, during testing the average temperature as delivered to a patient for two bags of fluids warmed by the disclosed warming pouch with respective starting temperatures of 5 and 20 °C and a 50 mL/min flow rate converged to respective average temperatures of about 34.7 and 37.4 °C, as measured at a catheter. That is to say, the initial 15-degree delta converged to approximately a 2.7-degree delta at the point of input to the patient. However, this convergence has limits with regard to safety and even higher initial fluid temperatures may lead to excessively warmed fluid from a patient perspective. To compensate in this situation, the operator may choose to activate only one of the two warming pockets (when the warming pouch has two warming pockets) when warming a fluid bag. For example, a saline bag warmed by the disclosed warming pouch with a single activated warming pocket can reach an average temperature of about 32.3 °C measured at a catheter, assuming a 23.5 °C starting temperature and a 50 mL/min flow rate. However, in certain cold climates, the ambient temperature may be closer to the recommended storage and transport temperature for blood. In such circumstances, the operator may still activate both warming pockets to achieve the desired amount of warming.

Because the entire fluid bag is warmed in a single operation, there is no requirement to prime an administrative or IV set. As a result, an operator can start the flow at any time. However, for optimal results, the recommended practice is to allow the disclosed warming pouch to complete the warming cycle while preparing the administration set and obtaining intravenous access.

In an embodiment, each pair of activation and warming pockets does not communicate with another pair of activation and warming pockets located on the other side of the warming pouch. In some embodiments, a warming pouch may contain only a single pair of activation and warming pockets. Such a warming pouch costs less to manufacture and to purchase, and may serve only to warm non-blood fluids from a typically higher initial temperature, for example.

In some embodiments, the reactant is contained within a warming pad to ease loading into the warming pocket during manufacture and to promote the rapid and even distribution of the activation solution throughout the reactant after activation. In an embodiment, the warming pad comprises two sheets of gas and water permeable material bonded to each other forming at least one chamber to house the reactant. In another embodiment, the warming pad comprises two sheets of water permeable woven fabric that is thermally bonded to form four vertical chambers.

It should be appreciated that other methods to contain, load, and saturate the reactant may be utilized, such as loading the reactant directly into a warming pocket of the warming pouch. Alternatively, a warming pad is constructed to comprise non-woven fabric thermally bonded multiple times in horizontal and vertical directions to create a checkerboard-like set of smaller chambers. Such a design might be complex but may promote more even mixing between an activation solution and an enclosed reactant.

In an embodiment, each warming pocket contains approximately 6 grams (“g”) of a powdered mixture of Mg—Fe alloy that serves as a reactant. In embodiments where the warming pouch includes an activation pocket, such a pocket contains approximately 18 milliliters (“mL”) of hypertonic saline to serve as an activation solution. It should be appreciated that different quantities of reactant and activation solution may be utilized depending on the application and environment. For example, in arctic operations, the need for increased warming may require an increase in both reactant and activation solution. Conversely, a warming pouch purely targeting the warming of non-cold stored fluids from ambient temperature in moderate temperature environments may require less reactant and activation solution.

The advantages of the chemistry between the reactant and the activation solution relate to energy and size: a relatively small volume and mass of powdered alloy and saline can generate enough energy to rapidly warm a unit of fluid in order to reduce the risk of hypothermia in a patient. This approach also has disadvantages, however. Specifically, during the several minutes of the warming cycle, the disclosed warming pouch generates and releases into the local environment approximately 1 liter (“L”) of hydrogen gas per gram of reactant at standard temperature and pressure. This is the approximate theoretical maximum. Due to inefficiencies in absorption, the actual rate may be approximately 0.75-0.90 L per gram. This amount of gas is typically not an issue for single-unit activations of the warming pouch due to the escape property of hydrogen. However, as larger numbers of the warming pouches are activated within an enclosed space over a short period, the byproduct hydrogen gas generated by the reaction must be vented, which inconveniences the operator and, if used in sufficient quantities in an enclosed area, can present a potential fire, explosive, and/or asphyxiation hazard.

As such, it should be appreciated that other chemistries to generate the exothermic reaction in the warming pouch may be utilized. In an embodiment, the strongest candidates based on heat released and safety are acid-based neutralization reactions. In an example, although the total mass would increase, water may be combined with either a mixture of aluminum chloride and calcium oxide (AlCl₃—CaO) or a mixture of diphosphorus pentoxide and calcium oxide (P₂O₅—CaO) to generate sufficient heat for the intended use without also generating hydrogen gas or other hazardous byproducts. Both chemistries can use similar packaging of the fluids and powdered mixtures in the embodiments discussed herein—including shipping without the activation solution included with the warming pouch. Such a development effort might be complex and time-consuming, but would nevertheless be a routine undertaking of engineering for those of ordinary skill in the art having the benefit of this disclosure.

In an alternate embodiment, to reduce the risk of accidental activation, the device may be manufactured without an activation pocket, activation solution, or a second frangible seal. To initiate the warming cycle, an operator instead manually adds the activation solution from an external source. In some embodiments, the activation solution accumulates in an activation pocket above a warming pocket containing the reactant. An operator causes the first frangible seal to break or breach, thereby allowing the accumulated activation solution to mix with the reactant.

In an example based on the disclosed Mg—Fe powdered mixture, an operator injects hypertonic saline from a syringe or squeeze bottle into one or more apertures that are located at the top of the warming pouch. In some instances, the apertures are also the vents configured to enable steam and gaseous byproducts generated from the exothermic reaction to be released. When the activation solution is added to the warming pouch, the fluid flows downward to mix with the reactant after the first frangible seal is broken. In an embodiment, the vents do not extend all the way through the bag, but instead only through at least one exterior wall to help direct the operator-added fluid into the warming pocket. In this embodiment, the vents are sized to allow for a tip of a luer lock-tipped syringe or squeeze bottle to inject saline fluid into the warming pouch.

In other embodiments, the first frangible seal (and the activation pocket) is also omitted from the warming pouch. In these other embodiments, the warming pouch receives the activation solution through the one or more apertures/vents. Without the first frangible seal, the activation solution flows downward between the interior and exterior walls to the warming pocket for mixing with the reactant.

In an alternate embodiment based on the Mg—Fe powdered mixture, to simplify logistics the device may be activated by the addition of isotonic saline instead of a specialized hypertonic saline. Isotonic saline is already a commonly carried provision of battlefield medics (and civilian EMTs) and, as such, its incorporation as the mechanism to initiate the warming cycle results in simplified logistics and operations. However, isotonic saline has a lower NaCl content than hypertonic saline. To make up for the lower NaCl concentration in isotonic saline, the difference in NaCl is added to the powdered mixture of the Mg—Fe alloy. As such, the disclosed warming pouch may be manufactured without the activation solution, the activation pocket, and the second frangible seal, and instead incorporates NaCL into the reactant contained within the activation pocket. In this embodiment, the vents do not extend all the way through the warming pouch, but instead only through the exterior wall in order to help direct the added fluid into the warming pocket. In this embodiment, the vents are sized to allow for a tip of a luer lock syringe flush kit (medics carry multiple flush kits into battle) to inject saline fluid into the warming pouch.

In an alternate embodiment based on the Mg—Fe powdered mixture, to further simplify logistics, the warming pouch may be activated by the addition of water instead of either isotonic or hypertonic saline. To make up for the absent NaCl, the difference in NaCl is added to the powdered mixture of the Mg—Fe alloy. As such, the warming pouch may be manufactured without the activation solution, the activation pocket, and the second frangible seal. Instead, the disclosed warming pouch incorporates the full amount of the NaCL otherwise omitted from the omitted hypertonic saline into the reactant contained within the activation pocket. In this embodiment, the vents do not extend all the way through the warming pouch, but instead only through at least one of the exterior walls to help direct the added fluid into the warming pocket. In this embodiment, the vents are sized to allow for a tip of a luer lock syringe flush kit to inject saline fluid into the warming pouch.

One of the largest components in the total cost of the disclosed warming pouch is the regulatory framework for the transportation and storage of products that contain a HAZMAT designation. In addition to the costs of testing and reporting for regulatory purposes, a HAZMAT designation imposes additional handling and shipping costs with commercial carriers. As such, to avoid a HAZMAT designation, in an alternate embodiment based on the Mg—Fe powdered mixture, instead of manually adding water or saline, the warming pouch may be activated by the manual addition of water and NaCl as separate components. Although water and NaCl can be combined into saline, separating them for purposes of activation allows for logistics to ship the warming pouch and the associated salt, which may be packetized, in separate packages to avoid the HAZMAT designation. These separate packages could be used to activate other warming pouches without meeting the HAZMAT designation, such as MREs. To use, an operator adds the salt to the reactant and then adds water from any source. As such, the device may be manufactured without the activation solution, the activation pocket, and frangible seal(s). Instead, the disclosed warming pouch incorporates one or more activation pockets with a powdered mixture of the Mg—Fe alloy. In this embodiment, the vents do not extend all the way through the warming pouch, but instead only through the exterior wall to help direct the added fluid and NaCl into the warming pocket. In this embodiment, the vents are sized to allow for a tip of a luer lock syringe flush kit to inject saline fluid into the warming pouch.

To mitigate the risk of fluid leaking during the warming cycle, such as by a warming pouch tipped sideways, in an embodiment, the warming pouch utilizes a gas-permeable membrane to allow air and hydrogen to escape through a top of the warming pouch, even when not held upright, but to keep liquid and water vapor trapped inside. In an example, this membrane forms a layer directly behind the vents.

In an embodiment, the warming pouch folds for storage. In an example, the pocket containing the activation pocket folds over the warming pocket, much like folding a man's billfold. Leakage of the activation solution causing unintended activation during transport and storage is a risk to be considered. To mitigate this risk, in an embodiment, the warming pouch is stored and transported with the crease of the fold pointed upwards during storage and transport, such that if the activation pocket is damaged, any leakage of activation solution flows downward and away from the device rather than into the warming pocket.

In another embodiment, the reactive chemistry described above is scaled up to provide recovery heat for a patient experiencing hypothermia. In an example, the reactive chemistry is contained in a warming blanket.

### Warming Pouch Embodiments

Referring now to the figures, FIGS. 1A and 1B show a fluid warming pouch 100, according to an example embodiment of the present disclosure. As disclosed herein, the fluid warming pouch 100 is configured to warm blood and other fluids. FIG. 1 shows a side view of the warming pouch 100, while FIG. 1B shows a top-down view looking into the warming pouch 100.

The warming pouch 100 includes a body 101 that has two sides 102a and 102b. The example sides 102a and 102b are bonded to each other along vertical edges 103, thereby forming a sleeve that defines a cavity 104 for receiving a container of fluid. In some embodiments, as shown in FIG. 1A, the two sides 102a and 102b are also bonded to each other along a portion of a bottom edge 103b. In some embodiments, the sides 102a and 102b may be integrally combined together to form a continuous tubular sleeve along a circumference of the warming pouch 100.

Each of the sides 102a and 102b is formed from an interior flexible wall 105 and an exterior flexible wall 106. The respective pair or set of interior flexible walls 105 and exterior flexible walls 106 are bonded to each other to create or form a warming pocket 107 therebetween for housing reactant 110. In the illustrated example of FIGS. 1A and 1B, the reactant 110 may be located in the warming pockets 107 formed in each of the sides 102a and 102b. The reactant 110 is chosen from the group of a mixture of Magnesium and Iron (Mg—Fe), a mixture of aluminum chloride and calcium oxide (AlCl₃—CaO), or a mixture of diphosphorus pentoxide and calcium oxide (P₂O₅—CaO). Further, the reactant may include similar sizes particles or particles of different sizes.

In some embodiments, the warming pouch 100 includes one side 102a with the interior flexible wall 105 and the exterior flexible wall 106. In these embodiments, an opposing side 102b includes a single flexible sheet. Alternatively, the opposing side 102b also includes the interior flexible wall 105 and the exterior flexible walls 106. However, the interior flexible wall 105 and the exterior flexible walls 106 of the opposing side 102b may not include reactant. Further, in this embodiment, tops of the interior flexible wall 105 and the exterior flexible walls 106 of the opposing side 102b may be bonded together.

As shown in FIG. 1A, each of the sides 102a and 102b includes at least one vent 108, which may be defined by an aperture in the body 101. Each of the vents 108 is fluidly coupled to one of the respective warming pockets 107. As disclosed above, the vents 108 enable steam and gaseous byproducts generated from an exothermic reaction involving the reactant and an activation solution. The vents 108 may also allow the activation solution to be inserted or injected into the respective warming pocket 107 for mixing with the reactant 110.

In some embodiments, a separate aperture 114 may be provided on an exterior of the external wall 106. The separate aperture 114 is configured to receive the activation solution. When used, the aperture 114 is sized to receive a nozzle for injecting the activation solution into the warming pouch 100.

In some embodiments, the warming pouch also includes a tab 109. As shown in FIG. 1A, the tab 109 is located at a top end of the warming pouch 100 when positioned for use. The tab 109 is connected to a top edge of the body 101 and defines an aperture to allow for receipt of a hook. While a single tab 109 is shown, in other embodiments the warming pouch 100 may include two or more tabs 109. Further, in some embodiments, the warming pouch 100 may omit the tab 109.

In addition to an opening at the top of the warming pouch 100 for receiving a container of fluid, the warming pouch 100 also includes a smaller opening 112 at a bottom end. The smaller opening 112 is configured to enable a connector or a port of the container to pass through for connection to an administration set or an IV line set. A portion of the bottom of the sides 102a and 102b may have a curved recess or cutout at the smaller opening 112.

In the illustrated embodiment, the warming pouch 100 omits seals. Instead, activation solution flows directly to the reactant 110 when added through one or more of the vents 108. Such a configuration enables a user to start the exothermic reaction without having to apply external pressure to break a seal.

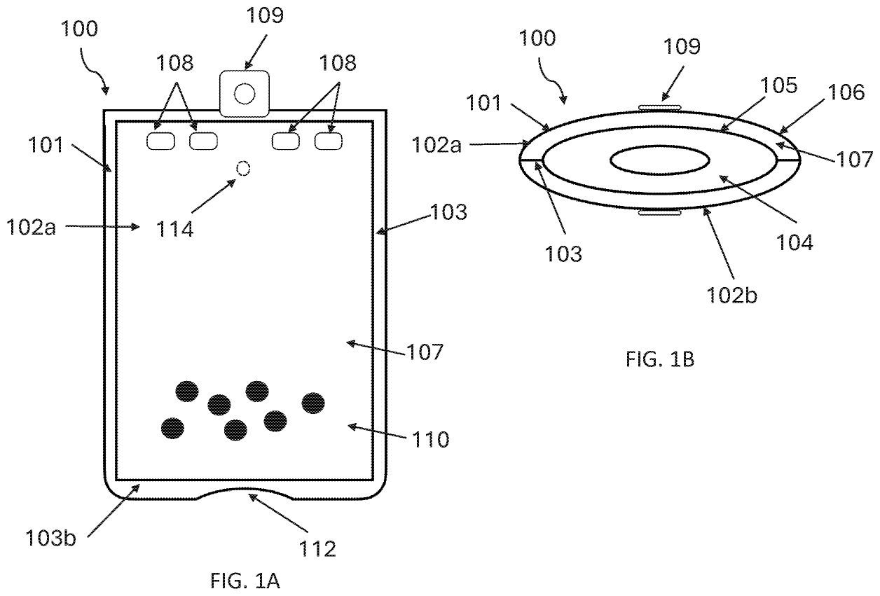

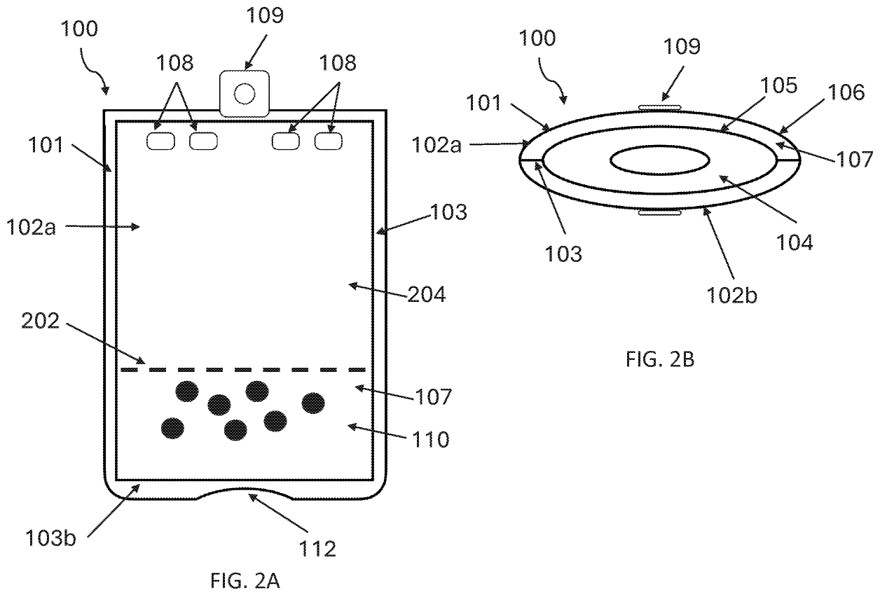

FIGS. 2A and 2B show an embodiment of the warming pouch 100 with a seal 202, according to an example embodiment of the present disclosure. In this embodiment, each of the sides 102a and 102b with the reactant 110 includes a seal 202. As shown, the seal 202 is located between the warming pocket 107 and an activation pocket 204. The seal 202 is a frangible seal that is configured to break under pressure.

In this embodiment, the activation pocket 204 is initially empty. A user has to first add fluid to the activation pocket 204 through one of the vents 108 or a port connected to one of the vents. The activation solution accumulates in the activation pocket 204. After pressure is applied, the seal 202 breaks, thereby creating a fluid coupling between the activation pocket 204 and the warming pocket 107. The fluid coupling enables the activation solution to mix with the reactant, thereby causing an exothermic reaction.

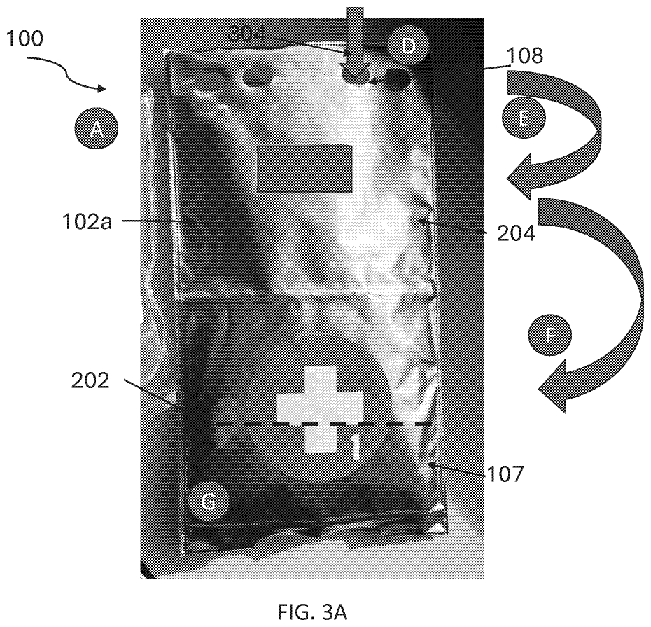

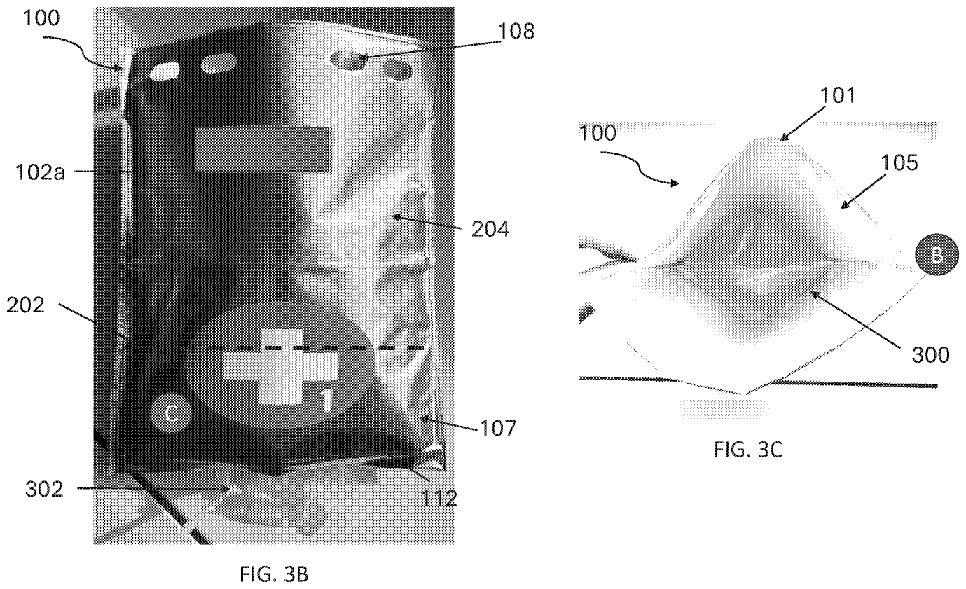

FIGS. 3A, 3B, and 3C show an example use of the warming pouch 100, according to an example embodiment of the present disclosure. At Event A, an operator obtains the warming pouch and a container of fluid 300 (shown in FIG. 3B). At Event B (shown in FIG. 3C), the operator opens the warming pouch 100 and inserts the container of fluid 300. The container of fluid 300 may include whole blood, a blood product, a crystalloid, a colloid, and/or water. The container of fluid 300 may be configured to hold 0.5 L of fluid. In other embodiments, the warming pouch 100 is configured to hold 0.25 L containers, 1 L containers, 2 L containers, 5 L containers, etc.

A top of the of the warming pouch 100 is large enough for receiving the container of fluid 300 within a cavity that is defined by a sleeve of the body of the warming pouch 100. At Event C (FIG. 3B), the operator pulls a connector or port 302 of the container of fluid 300 through the smaller opening 112 for connection to an administration set or IV line set. In some instances, the user may hold off connecting the administration set until after the exothermic reaction has started.

At Event D, the operator adds an activation solution 304 through one or more of the vents 108. The activation solution 304 may include water, isotonic saline, hypertonic saline, and an antifreeze additive. When the antifreeze additive is used, the antifreeze additive may be calcium chloride (CaCL₂). Further, in some embodiments, the activation solution is provided as a 28% calcium chloride solution.

The activation solution 304 accumulates in the activation pocket 204. After the activation solution 304 has been added, the operator folds over a top portion of the warming pouch 100, as shown at Event E in FIG. 3A. Folding over the top portion of the warming pouch 100 creates a fluid-tight seal or kink between the activation pocket 204 and the vent(s) 108. This seal prevents the activation solution 304 from exiting the vent(s) 108 when pressure is applied to break the seal 202.

In alternative embodiments, the warming pouch 100 includes a closable seal located between the vent(s) 108 and the activation pocket 204. The closable seal may include a zip seal or a press seal. After the activation solution 304 is added, the operator may close the seal to prevent the activation solution 304 from escaping when pressure is applied to break the seal 202.

At Event F, pressure is applied to the seal 202. Pressure may be applied by folding a top half or more of the warming pouch 100 down toward the warming pocket 107 and pressing the halves together. Alternatively, an operator may apply force to the activation pocket 204, thereby compressing the activation solution 304, causing it to apply pressure and eventually break the seal 202. In alternative embodiments, the seal 202 may include a peelable seal that is removed by peeling away an adhesive barrier.

After the seal 202 is broken, a channel is created to enable the activation solution 308 to mix with the reactant 110 at Event G, thereby starting an exothermic reaction. This begins a period of rapid energy release (e.g., the “warming cycle”) to reach a minimum target temperature. It should be appreciated that in some embodiments, the container of fluid 300 is added only after the exothermic reaction begins.

In an embodiment, the configuration of the reactant and the activation solution causes the warming cycle to last five minutes or less to cause the container of fluid 300 reach a target temperature between 30 °C and 46 °C, preferably around 37 °C. In another embodiment, the configuration of the reactant and the activation solution causes the warming cycle to last two to three minutes and reach a minimum target temperature of approximately 20 °C. While faster heating and/or higher minimum target temperatures are technically possible, in the case of whole blood and blood products, achieving such goals increases the risk of causing damage by application of excessive heat to the fluid in contact with the walls of the fluid bag. Conversely, warming cycles longer than two to three minutes create the risk of damage to a patient by delaying treatment. Thus, for whole blood and product products, while a wide range of values are technically possible, for battlefield and many other purposes an ideal time range for the warming cycles is between two to three minutes, which has the additional benefit of being the typical amount of time needed by an operator to prepare an administrative set and obtain intravenous access.

Heat generated from the exothermic reaction propagates through the internal walls 105 of the body 101 to the container of fluid 300. The body 101 of the warming pouch may direct heat towards the container of fluid 300 by having a greater thermal conductivity at the point that touches the container 300. In some embodiments, the interior wall 105 may touch the container of fluid 300. The interior wall 105 may be composed of a different material than the exterior wall 106, such that the interior wall has a larger thermal conductivity than the exterior wall, increasing the heat flow towards the container of fluid 300. The exterior wall 106 may have a low thermal conductivity to mitigate the risks of burning to a user while the exothermic reaction is ongoing. In some examples, the body 101 (including the walls 105 and 106) may be composed of a plastic, a foil, a plasticized foil, a laminated foil, or a combination thereof.

During the warming cycle, steam and other gaseous byproducts escape from the warming pouch 100 through the one or more vents 108(s). After the exothermic reaction begins, the operator unfolds the warming pouch 100 to enable the steam and other gaseous byproducts to reach the vent(s). Venting the steam and other gaseous byproducts prevents extraneous pressure from building up inside the warming pouch 100, which could apply pressure to the container of fluid 300, thereby causing the fluid to flow too quickly.

After the warming cycle completes, the warming pouch 100 transitions from rapid warming to instead expending a lower amount of energy to maintain the temperature of the container of fluid 300 within a target range (the “runtime cycle”). In some embodiments, the warming pouch 100 may produce negligible amounts of heat during the runtime cycle. In such embodiments, the fluid within the container of fluid 300 may stay within the target range due primarily to residual heat generated during the runtime cycle and communicated from the warming pouch to the fluid bag during the warming cycle.

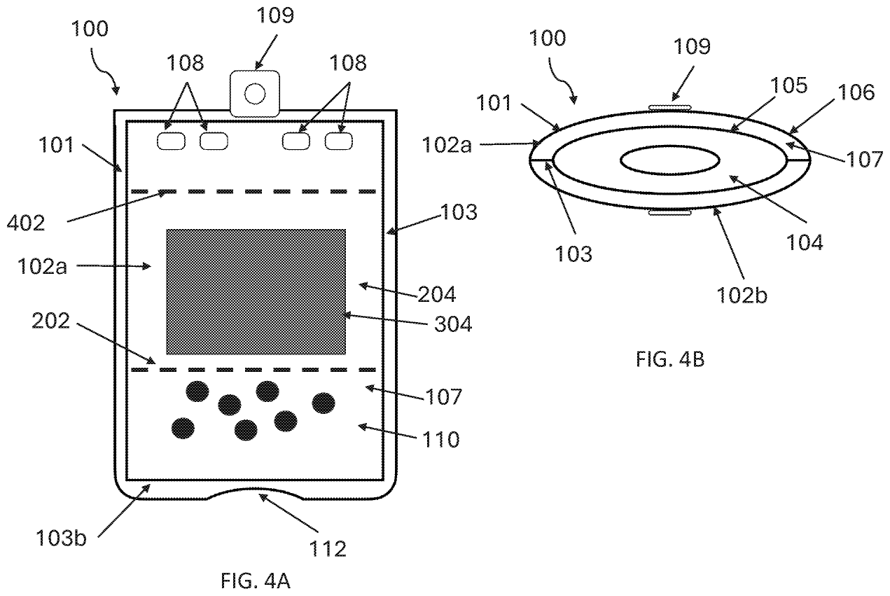

FIGS. 4A and 4B show an embodiment of the warming pouch 100 where the activation solution 304 is sealed in the activation pocket 204 at the time of manufacture, according to an example embodiment of the present disclosure. In this embodiment, the warming pouch 100 includes a second seal 402, which is located between the activation pocket 204 and the one or more vents 108.

As disclosed above, the seal 202 may be positioned between the warming pocket 107 and the activation pocket 204. The seal 202 may be configured to break within a first range of pressures from 0.25 psi to 2 psi, preferably between 0.5 psi and 1 psi. In some embodiments, the first range of pressures may include pressure an operator could manually apply to the seal 202. As discussed above, in some examples, the operator may break the seal 202 by folding the body at the activation pocket 204, decreasing the respective volume of the activation pocket 204 until the activation solution 304 breaks through the frangible seal 202. In embodiments where the warming pouch 100 takes the form of a sleeve, the sleeve may be unconnected at a top portion of the warming pouch 100 to allow for the folding discussed in the previous example. An operator may fold the body 101 of the warming pouch 100 away from a center axis of the sleeve and press downwards to break through the frangible seal 202. In some examples, approximately one and three quarters inches of the sleeve remains unconnected at the top portion.

The second seal 402 may be positioned between the vent 108 on one side 102a/102b and the warming pocket 107, the activation pocket 204, or a combination thereof on the other side. The second seal may be configured to break within a second range of pressures. In some embodiments, the second range of pressures may be between 0.1 psi to 1 psi. In some embodiments, the second range of pressures may be relative to the first range of pressures, where the second range of pressures is 10% to 50% less than the first range of pressures, preferably 25% less than the first range of pressures. In some embodiments, the second range of pressures may include the increased pressure from the exothermic reaction. In some embodiments, the second range of pressures may be lower than the first range of pressures. In such embodiments, the second seal 402 becomes easier to break than the first seal 202, mitigating the risks of accidentally activating the warming pouch 100 prematurely. Instead, the second seal 402 may rupture first, allowing for the activation solution 304 to drain from the warming pouch 100 prior to entering the warming pouch, preventing the exothermic reaction from occurring accidentally. The second seal 402 may be air and liquid tight prior to rupturing to prevent any leakage of the activation solution 304.

The vent 108 may be the first of a plurality of vents. In some embodiments, there may be an equal number of vents to the number of warming pockets. In further embodiments, there may be an equal number of vents 108 to the number of chambers in a warming pad, as discussed below. The vent 108 may be defined by an aperture in the body 101 of the warming pouch 100. In some embodiments, the vent 108 may be defined by an aperture formed between an unbonded portion of the interior wall 105 and the exterior wall 106. In some embodiments, the vent 108 may be die-cut within the body 101. The vent may run through the interior wall 105, the exterior wall 106, or a combination thereof. In one embodiment, the vent 108 may be formed as a cutout in the exterior flexible wall 106. In another embodiment, the vent 108 may be formed as a cutout in the interior flexible wall 105. Preferably, the vent 180 is formed with a cutout in the exterior flexible wall 106 to allow venting away from an interior of the warming pouch 100. The vent 108 may be fluidically coupled to the pockets 107 and 204 defined between the interior wall 105 and the exterior wall 106. For illustrative purposes, the fluidically coupling is represented by a channel between the pockets 107 and 204 and the vents 108. The vents 108 define a path from the pockets 107 and 204 to the exterior atmosphere. Each vent 108 may be positioned such that the vent is above the warming pocket 107 and activation pocket 204 while the warming pouch 100 is in use, such that the rising steam and gaseous byproducts organically flow through the vent 108.

In the illustrated example, the activation pocket 204 is sized to accommodate the activation solution 304. According to some embodiments, the activation pocket 204 may be sized proportionally to the warming pocket 107. In some examples, the activation pocket 204 and the warming pocket 107 may be approximately equal in size. In some examples, the activation pocket 204 may range from 100% to 50% the size of the warming pocket 107. In some examples, the activation pocket 204 may range from 85% to 70% the size of the warming pocket 107. According to some embodiments, the warming pouch 100 may include a plurality of activation pockets 204. The body 101 may define channels fluidically connecting the plurality of activation pockets 204.

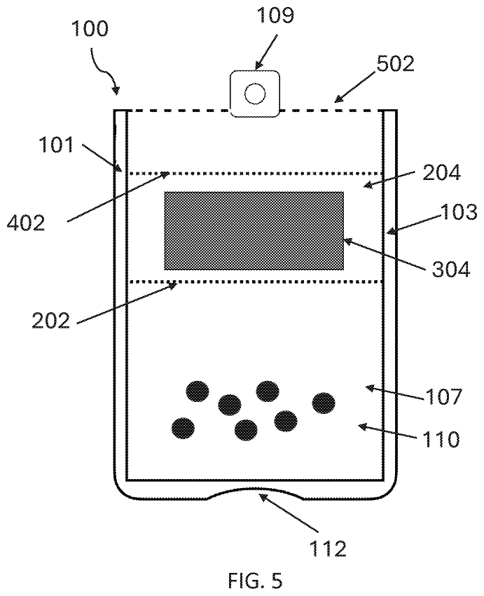

FIG. 5 shows an embodiment of the warming pouch 100 where the vents 108 are removed, according to an example embodiment of the present disclosure. Instead, a top end 502 of the warming pouch 100 is open to the second seal 402 (or the activation pocket 204 when the second seal 402 is not present). At the top end 502, the interior wall 105 and the exterior 106 are not bonded. Such a configuration of the warming pouch 100 enables the steam and other gaseous byproducts to escape directly upward.

### Warming Pad Embodiment

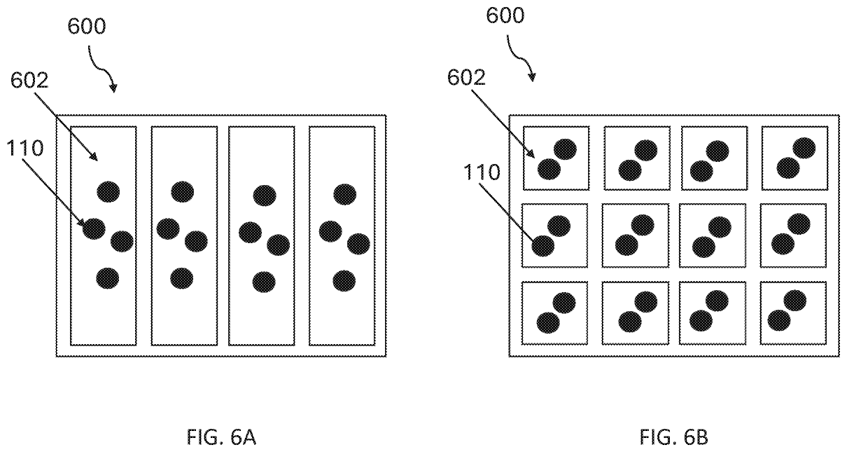

As discussed above, the reactant 110 is contained within the warming pocket 107 of the warming pouch 100. In some embodiments, the reactant 110 is enclosed in a removable warming pad 600, as shown in FIGS. 6A and 6B, according to an example embodiment of the present disclosure. The warming pad 600 may include a plurality of sheets of gas and water permeable materials bonded to each other defining at least one chamber 602 between the two sheets. The reactant 110 may be housed within the chamber 602 of the warming pad 600. The warming pad 600 may be one of a plurality of interchangeable warming pads, allowing for reusability of the warming pouch 100. Alternatively, the plurality of interchangeable warming pads 600 may provide adaptability based upon user based needs regarding the shape, material, reactant used, or other criteria as the user sees fit. The warming pocket 107 may be sized to accommodate the warming pad 600.

FIG. 6A shows a warming pad 600 with four parallel chambers 602. When activation solution 304 flows downward, each of the chambers 602 receive the activation solution substantially equally. FIG. 6B shows a warming pad 600 with chambers 602 arranged in a grid pattern. It should be appreciated that the chambers 602 may be arranged in any number of ways, including a checkerboard pattern, a hexagonal pattern, etc.

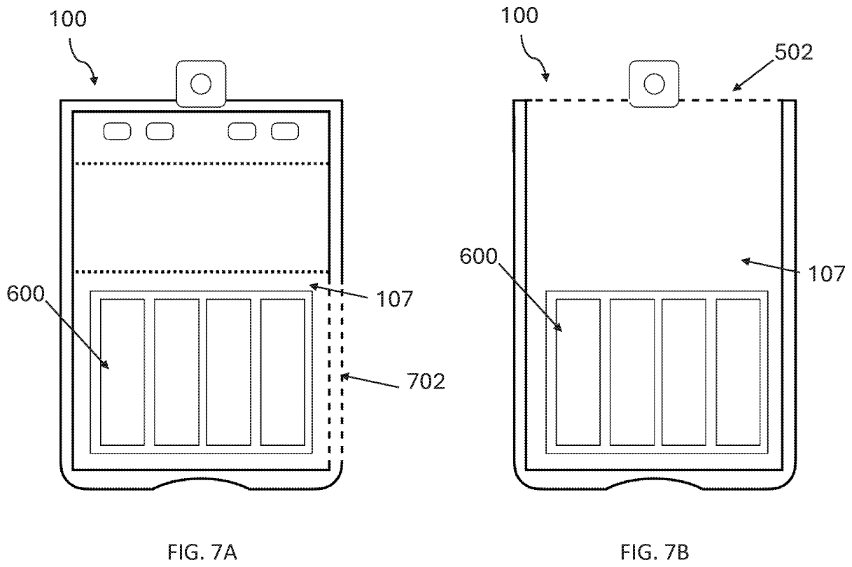

FIGS. 7A and 7B show different ways of inserting the warming pad 600 into the warming pouch 100, according to example embodiments of the present disclosure. As shown in FIG. 7A, a side of the warming pocket 107 is open for receiving the warming pad 600. The warming pad 600 may be inserted, for example, through a slit 702 in the exterior wall 106 of the body 101 of the warming pouch 100. The slit 702 may then be sealed after the warming pad 600 has been inserted. In some instances, the slit 702 may configured to be openable by an operator to replace the warming pad 600 after use.

As shown in FIG. 7B, the warming pad 600 is inserted through the top end 502 of the warming pouch 100. The warming pad 600 is lowered through the warming pouch 100 until reaching the warming pocket 107. In some embodiments, the seal 202 and/or the seal 402 may then be formed between the interior wall 105 and the exterior wall 106.

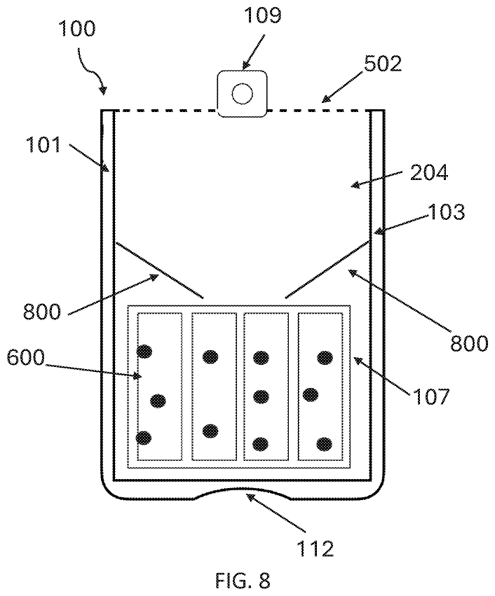

In an alternative embodiment, the seals 202 and 402 may be removed when the warming pad 600 is used with the warming pouch 100. In these embodiments, the warming pad 600 is configured to retain the reactant in place. FIG. 8 shows an embodiment where the warming pouch 100 instead includes partitions 800, according to an example of the present disclosure. The example partitions 800 are configured to keep the warming pad 600 in place. In addition, the partitions 800 are configured to enable the activation solution to flow to the warming pad 600 and enable steam and other gaseous byproducts to escape from the warming pouch 100.

In the illustrated example, the partitions 800 are connected to interior edges 103 of the exterior wall 106 and the internal wall 105. The partitions 800 are angled downward for directing the flow of the activation solution to the warming pad 600. In some embodiments, the partitions 800 form a funnel for direction the activation solution when the warming pouch 100 is position for use.

### Conclusion

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A fluid warming pouch comprising:
a body having a plurality of sides, each side comprising an interior flexible wall configured to abut a container of fluid and an exterior flexible wall, wherein the interior flexible wall and the exterior flexible wall are bonded to each other, at least one of the plurality of sides including:
a warming pocket housing a reactant,
an activation pocket housing an activation solution, wherein the activation pocket is fluidically coupled to the warming pocket,
a first frangible seal positioned between the warming pocket and the activation pocket, wherein breakage of the first frangible seal creates a channel between the warming pocket and the activation pocket allowing for intermixing of the reactant and the activation solution to generate heat via an exothermic reaction,
a vent to an exterior atmosphere, wherein the vent is fluidically coupled to at least one of the warming pocket, the activation pocket, or a combination thereof, and
a second frangible seal positioned between the vent and the at least one of the warming pocket, the activation pocket, or a combination thereof, wherein breakage of the second frangible seal creates a channel between the at least one of the warming pocket, the activation pocket, or a combination thereof and the exterior atmosphere allowing for gaseous byproducts of the exothermic reaction to escape to the exterior atmosphere.

2. The fluid warming pouch of claim 1, wherein the warming pocket is a first warming pocket of a plurality of warming pockets housing the reactant.

3. The fluid warming pouch of claim 1, wherein the warming pocket and the activation pocket are depressurized pockets.

4. The fluid warming pouch of claim 1, wherein the activation pocket is a first activation pocket of a plurality of activation pockets that house the activation solution.

5. The fluid warming pouch of claim 1, wherein the warming pocket contains a warming pad, the warming pad comprising:
a chamber comprising a plurality of sheets of gas and water permeable material bonded to each other, wherein the chamber houses the reactant.

6. The fluid warming pouch of claim 5, wherein the chamber is a first chamber in a plurality of chambers.

7. The fluid warming pouch of claim 1, wherein the first frangible seal is configured to break within a first range of pressures.

8. The fluid warming pouch of claim 7, wherein the second frangible seal is configured to break within a second range of pressures, wherein the second range of pressures is lower than the first range of pressures.

9. The fluid warming pouch of claim 1, wherein the container of fluid contains at least one of whole blood, a blood product, a crystalloid, a colloid, or water.

10. The fluid warming pouch of claim 1, wherein the reactant is a powder comprising a plurality of particulate sizes.

11. The fluid warming pouch of claim 1, wherein the body is composed of plastic, foil, plasticized foil, laminated foil, or a combination thereof.

12. The fluid warming pouch of claim 1, wherein the interior flexible wall is composed of a material with a first thermal conductivity, and wherein the exterior flexible wall is composed of a material with a second thermal conductivity, wherein the first thermal conductivity is greater than the second thermal conductivity.

13. A method of warming a fluid contained in a container, the method comprising:
providing a warming pouch including:
a body defining a cavity configured to receive the container with the fluid,
a reactant disposed within a warming pocket of the body,
an activation pocket configured to receive an activation solution,
a frangible seal separating the activation pocket from the reactant, and
one or more vents in fluid communication with the activation pocket;
receiving the container in the cavity of the warming pouch;
receiving the activation solution in the activation pocket through the one or more vents;
receiving pressure at the activation pocket to break the frangible seal and cause the activation solution to mix with the reactant;
causing, in response to the mixing, an exothermic reaction that generates heat to begin, thereby starting a warming cycle;
causing the generated heat to transfer through an interior wall of the warming pouch to the container to raise a temperature of the fluid to a target temperature; and
venting gaseous byproducts of the exothermic reaction through the one or more vents to limit pressure buildup within the warming pouch.

14. The method of claim 13, wherein introducing the activation solution comprises introducing the activation solution in a volume sufficient to initiate the exothermic reaction within less than five minutes.

15. The method of claim 13, wherein the activation solution comprises water, hypertonic saline, or isotonic saline.

16. The method of claim 13, wherein the activation solution comprises an aqueous calcium chloride solution.

17. The method of claim 16, wherein the aqueous calcium chloride solution has a concentration of about 28% by weight.

18. The method of claim 13, wherein the reactant is a powder comprising a plurality of particulate sizes.

19. The method of claim 13, wherein the warming cycle is configured to raise the temperature of the fluid to the target temperature within five minutes or less.

20. The method of claim 13, wherein the warming cycle raises the temperature of the fluid to a temperature between about 30 °C and about 46 °C.
